(19) United States
(12) Patent Application Publication
(10) Pub. No.: US 2022/0252591 A1
(43) Pub. Date: Aug. 11, 2022

(54) LIQUID SAMPLE TESTING DEVICE

(71) Applicant: TANAKA KIKINZOKU KOGYO K.K., Tokyo (JP)

(72) Inventors: Soichiro SEKINE, Kanagawa (JP); Hiroshi KISHI, Kanagawa (JP); Daisuke ITO, Kanagawa (JP)

(73) Assignee: TANAKA KIKINZOKU KOGYO K.K., Tokyo (JP)

(21) Appl. No.: 17/629,176

(22) PCT Filed: Jul. 22, 2020
(86) PCT No.: PCT/JP2020/028555

§ 371 Date: Jan. 21, 2022

(30) Foreign Application Priority Data
Jul. 23, 2019 (JP) 2019-135727
Jul. 1, 2020 (JP) 2020-113853

(51) Int. Cl. G01N 33/543

(52) U.S. Cl. CPC G01N 33/54386

(57) ABSTRACT

A liquid sample testing device includes: an upper case having: a hole allowing a liquid sample to be dropped in the hole; and a window to determine a test result, the hole and the window being located beside each other; a lower case assembled to the upper case; and a test strip housed between the upper case and the lower case. The upper case has: a first protrusion protruding toward the lower case; and a second protrusion protruding toward the lower case. The first protrusion and the second protrusion each has a predetermined shape to allow a capillary force generated between the second protrusion and the liquid sample to be larger than a capillary force generated between the first protrusion and the liquid sample.

### TECHNICAL FIELD

The present invention relates to a liquid sample testing device.

### BACKGROUND ART

In recent years, liquid sample testing devices using immuno-chromatography have been frequently used especially for testing infectious diseases that are required to be promptly diagnosed. Such a type of liquid sample testing device includes a test strip, and an upper case and a lower case between which the test strip is housed. The liquid sample testing device has a configuration in which due to addition of a liquid sample to the test strip through a dropping hole provided in the upper case, a color indicating a test result can be visually recognized through a determination window that is provided to be located horizontally with the dropping hole.

In order to make it possible to visually recognize the test result promptly, the liquid sample that has been added through the dropping hole needs to be promptly spread to the determination window. Therefore, there has been considered a liquid sample testing device in which protrusive streaks are provided on a determination window side of the dropping hole of the upper case so that the liquid sample can be spread to the determination window side along the protrusive streaks (PTL 1).

### CITATION LIST

### Patent Literature

PTL 1: JP 4643415 B

### SUMMARY OF INVENTION

### Technical Problem

However, in the aforementioned existing liquid sample testing device, the liquid sample is promptly spread to the determination window side by the protrusive streaks. For this reason, the liquid sample flows toward the determination window at a stroke. That is, there is a problem that the liquid sample cannot be spread uniformly over the entire test strip. In addition, when the liquid sample cannot be spread uniformly, a dry medicine held by the test strip cannot be dissolved sufficiently. Therefore, there is another problem that reaction efficiency of immune reaction deteriorates, and formation of a color indicating a test result is poor.

The present invention has been accomplished in view of the aforementioned circumstances. An object of the present invention is to provide a liquid sample testing device in which a liquid sample can be spread uniformly over an entire test strip, and which improves efficiency of immune reaction and improves formation of a color indicating a test result.

### Solution to Problem

In order to achieve the aforementioned object, the liquid sample testing device according to the present invention is characterized by the following items [1] to [9].
[1] A liquid sample testing device comprising:

an upper case having: a hole allowing a liquid sample to be dropped in the hole; and a window to determine a test result, the hole and the window being located beside each other;

a lower case assembled to the upper case; and

a test strip housed between the upper case and the lower case,

the upper case having: a first protrusion protruding toward the lower case from a peripheral portion of the hole, the first protrusion being located on a side near to the window of the hole; and a second protrusion protruding toward the lower case from the peripheral portion of the hole, the second protrusion being located on a side opposite to the window of the hole compared to the first protrusion,

the first protrusion and the second protrusion each having a predetermined shape to allow a capillary force generated between the second protrusion and the liquid sample to be larger than a capillary force generated between the first protrusion and the liquid sample.
[2] The liquid sample testing device according to the item [1], wherein

the first protrusion extends from the peripheral portion of the hole toward the window along an arranged direction of the hole and the window,

the second protrusion extends from the peripheral portion of the hole to separate from the window along the arranged direction.
[3] The liquid sample testing device according to the item [2], wherein

a length of the second protrusion in the arranged direction is longer than a length of the first protrusion in the arranged direction.
[4] The liquid sample testing device according to the item [2] or the item [3], wherein

a plurality of the second protrusions are larger in number than a plurality of the first protrusions.
[5] The liquid sample testing device according to any one of the item [2] to the item [4], wherein

a plurality of the first protrusions are arranged side by side in a direction different from the arranged direction, and a plurality of the second protrusions are arranged side by side in a direction different from the arranged direction,

an interval between adjacent pair of the second protrusions is narrower than an interval between adjacent pair of the first protrusions.
[6] The liquid sample testing device according to the item [1], wherein

the second protrusion has a pillar shape,

a plurality of the second protrusions are aligned at intervals from the peripheral portion of the hole to separate from the window along an arranged direction of the hole and the window, and the plurality of the second protrusions form a second protrusion row,

a plurality of the second protrusion rows are arranged side by side in a direction different from the arranged direction.
[7] The liquid sample testing device according to the item [6], wherein

the first protrusion extends from the peripheral portion of the hole toward the window along the arranged direction,

a plurality of the first protrusions are arranged side by side in a direction different from the arranged direction,

an interval between adjacent pair of the second protrusions is narrower than an interval between adjacent pair of the first protrusions.
[8] The liquid sample testing device according to any one of the item [1] to the item [7],

the test strip has: a sample pad exposed in the hole; an adjusting pad located on a side near to the window of the sample pad with a gap between the sample pad and the adjusting pad; a conjugate pad located on a side near to the window of the adjusting pad, the conjugate pad touches to the adjusting pad; and a membrane located on a side near to the window of the conjugate pad, the membrane touches to the conjugate pad, the membrane is visually recognized through the window,

an end portion of the first protrusion on a side near to the hole in an arranged direction of the hole and the window is located above the sample pad, and an end portion of the first protrusion on a side near to the window in the arranged direction is located above the adjusting pad.
[9] The liquid sample testing device according to any one of the item [1] to the item [8],

the test strip has: a sample pad exposed in the hole; an adjusting pad located on a side near to the window of the sample pad with a gap between the sample pad and the adjusting pad; a conjugate pad located on a side near to the window of the adjusting pad, the conjugate pad touches to the adjusting pad; and a membrane located on a side near to the window of the conjugate pad, the membrane touches to the conjugate pad, the membrane is visually recognized through the window,

the first protrusion faces to the gap without being inserted into the gap.

According to the liquid sample testing device having the aforementioned configuration [1], the shapes of the first protrusions and the second protrusions are provided so that the capillary force generated between the second protrusions and the liquid sample can be made larger than the capillary force generated between the first protrusions and the liquid sample. Thus, the force with which the second protrusions pull the liquid sample that has been dropped on the test strip toward the opposite side to the determination window can be made stronger than the force with which the first protrusions pull the liquid sample that has been dropped on the test strip toward the determination window. Therefore, the flow of the liquid sample in the test strip can be controlled so that the liquid sample that has been dropped in the dropping hole is once moved forward to the opposite side to the determination window and then spread to the determination window side. Thus, the liquid sample can be spread uniformly over the entire test strip. In addition, due to the uniform spread of the liquid sample, a dry medicine held by the test strip can be sufficiently dissolved to improve efficiency of immune reaction and improve formation of a color indicating a test result.

According to the liquid sample testing device having the aforementioned configuration [2], the first protrusions and the second protrusions can be provided easily.

According to the liquid sample testing device having the aforementioned configuration [3], the length of each of the second protrusions in the arranged direction is longer than the length of each of the first protrusions in the arranged direction. Thus, the force with which the second protrusions pull the liquid sample that has been dropped on the test strip toward the opposite side to the determination window can be made stronger than the force with which the first protrusions pull the liquid sample that has been dropped on the test strip toward the determination window.

According to the liquid sample testing device having the aforementioned configuration [4], the number of the second protrusions is larger than the number of the first protrusions. Thus, the force with which the second protrusions pull the liquid sample that has been dripped on the test strip toward the opposite side to the determination window can be made stronger than the force with which the first protrusions pull the liquid sample that has been dripped on the test strip toward the determination window.

According to the liquid sample testing device having the aforementioned configuration [5], the interval between the adjacent second protrusions in the other direction than the arranged direction is narrower than the interval between the adjacent first protrusions. That is, a capillary path formed between the adjacent second protrusions is narrower than a capillary path formed between the adjacent first protrusions. Thus, the capillary force generated between the second protrusions and the liquid sample can be made larger than the capillary force generated between the first protrusions and the liquid sample.

According to the liquid sample testing device having the aforementioned configuration [6], the second protrusion rows in each of which a plurality of second protrusions are aligned in the arranged direction are provided to be arranged side by side in the other direction than the arranged direction. Thus, each of the second protrusions can be provided in the pillar shape.

According to the liquid sample testing device having the aforementioned configuration [7], the interval between the adjacent second protrusions is narrower than the interval between the adjacent first protrusions. That is, the capillary path formed between the adjacent second protrusions is narrower than the capillary path formed between the adjacent first protrusions. Thus, the capillary force generated between the second protrusions and the liquid sample can be made larger than the capillary force generated between the first protrusions and the liquid sample.

According to the liquid sample testing device having the aforementioned configuration [8], the end portions of the first protrusions on the dropping hole side in the arranged direction are located above the sample pad, and the end portions of the first protrusions on the determination window side in the arranged direction are located above the adjusting pad. Thus, the liquid sample that has been spread up to the end portions on the determination window side along the first protrusions can be infiltrated into the adjusting pad.

According to the liquid sample testing device having the aforementioned configuration [9], the first protrusions are provided to face the gap between the sample pad and the adjusting pad. Thus, the liquid sample can flow along the first protrusions to climb over the gap so as to be spread up to the adjusting pad.

### Advantageous Effects of Invention

According to the liquid sample testing device according to the present invention, as described above, it is possible to uniformly spread the liquid sample over the entire test strip. In addition, due to the uniform spread of the liquid sample, a dry medicine held by the test strip can be sufficiently dissolved to improve efficiency of immune reaction and improve formation of a color indicating a test result.

The present invention has been briefly described above. Furthermore, since modes for carrying out the invention (hereinafter referred to as “embodiments”) which will be described below will be read through with reference to the accompanying drawings, details of the present invention will be made clearer.

### BRIEF DESCRIPTION OF DRAWINGS

FIG. 1 is a perspective view of an upper case of a liquid sample testing device according to a first embodiment of the present invention.

FIG. 2 is a perspective view of a lower case of the liquid sample testing device according to the first embodiment of the present invention.

FIG. 3 is a sectional view of the liquid sample testing device according to the first embodiment of the present invention.

FIG. 4 is an enlarged perspective view of a key part of the upper case shown in FIG. 1.

FIG. 5 is a perspective view of an upper case of a liquid sample testing device according to a second embodiment of the present invention.

FIG. 6 is an enlarged perspective view of a key part of the upper case shown in FIG. 5.

FIG. 7 is a perspective view of an upper case of a liquid sample testing device according to a third embodiment of the present invention.

FIG. 8 is an enlarged top view of a key part of the upper case shown in FIG. 7.

### DESCRIPTION OF EMBODIMENTS

Specific embodiments relating to the present invention will be described below with reference to the respective drawings.

### First Embodiment

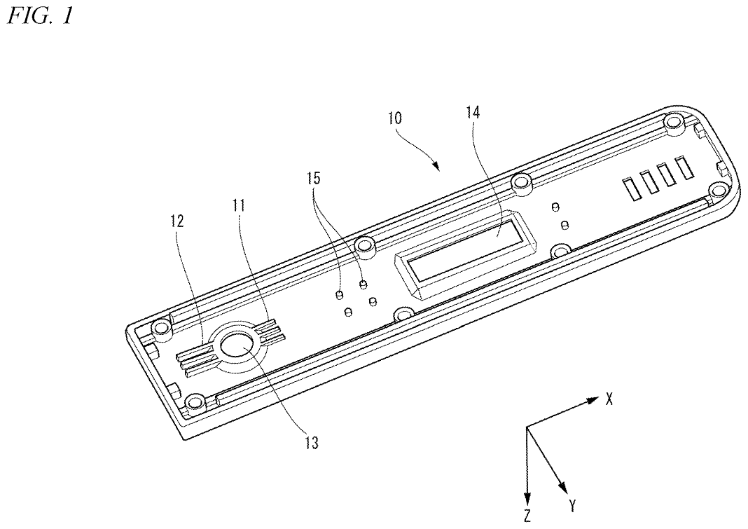

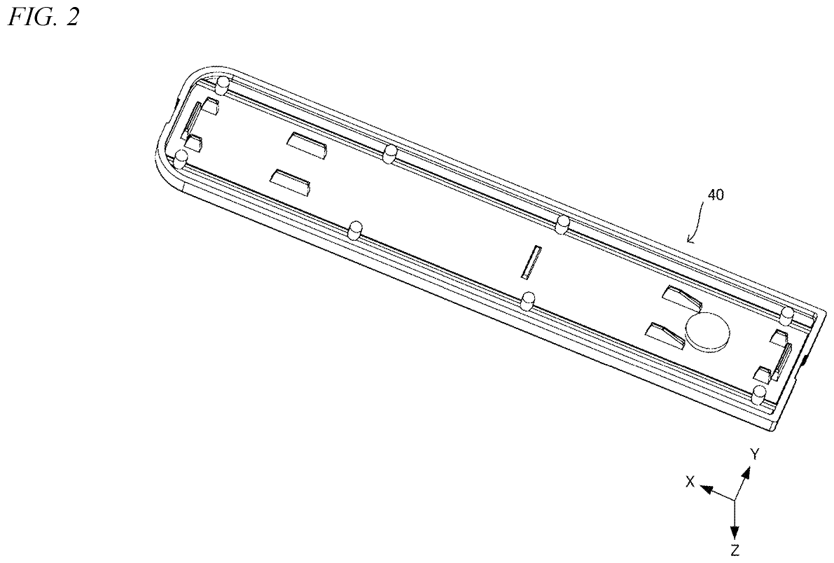

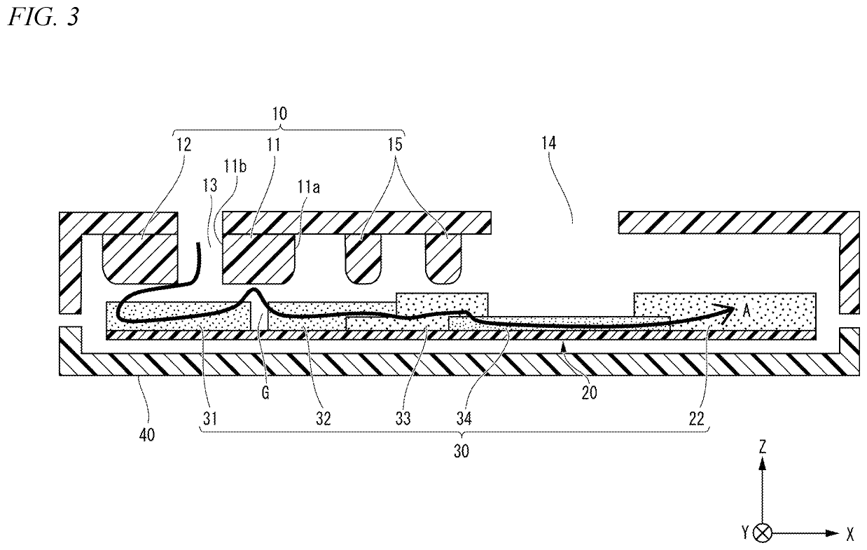

FIG. 1 shows a perspective view of an upper case 10 of a liquid sample testing device according to a first embodiment of the present invention. FIG. 2 shows a perspective view of a lower case 40 of the liquid sample testing device according to the first embodiment of the present invention. FIG. 3 shows a sectional view of the liquid sample testing device according to the first embodiment, particularly a sectional view of the upper case 10, the lower case 40, and a test strip 30. The liquid sample testing device according to the first embodiment includes the upper case 10 in which a hole 13 for dropping a liquid sample and a window 14 for determining a test result are provided to be arranged, the lower case 40 that is assembled to the upper case 10, and the test strip 30 that is housed between the upper case 10 and the lower case 40. The liquid sample testing device is, for example, a tool that uses immuno-chromatography for testing various infectious diseases, and has a configuration in which due to addition of the liquid sample to the test strip 30 through the dropping hole 13 provided in the upper case 10, a color indicating a test result can be visually recognized through the determination window 14 provided to be arranged horizontally with the dropping hole 13.

The upper case 10 has first protrusions 11, second protrusions 12, the dropping hole 13, the determination window 14, and pressing pins 15. FIG. 1 shows a perspective view of a back side of the upper case 10, that is, the perspective view of the upper case 10 when seen from a lower side of FIG. 3. The first protrusions 11, the second protrusions 12 and the pressing pins 15 protrude toward the test strip 30 placed on the lower case 40. On the other hand, the dropping hole 13 and the determination window 14 are provided so as to penetrate the upper case 10 so that the test strip 30 inside the liquid sample testing device can be visually recognized. In the present embodiment, the dropping hole 13 has a circular shape, and the determination window 14 has a rectangular shape. However, the respective shapes, the positions, and the like of the dropping hole 13 and the determination window 14 are not particularly limited.

Each of the first protrusions 11 is provided so as to protrude toward the lower case 40 and extend from a peripheral portion of the dropping hole 13 on the determination window 14 side along a direction X in which the dropping hole 13 and the determination window 14 are arranged. A front end of the first protrusion 11 faces the test strip 30 in a thickness direction Z. On the other hand, each of the second protrusions 12 is provided so as to protrude toward the lower case 40 and extend along the arranged direction X from a peripheral portion of the dropping hole 13 on an opposite side to the determination window 14. A front end of the second protrusion 12 faces the test strip 30 in the thickness direction Z. In FIG. 3, the first protrusion 11 and the second protrusion 12 are positioned at predetermined distances from the test strip 30, but the first protrusion 11 and the second protrusion 12 may contact the test strip 30. Details of the first protrusion 11 and the second protrusion 12 will be described below.

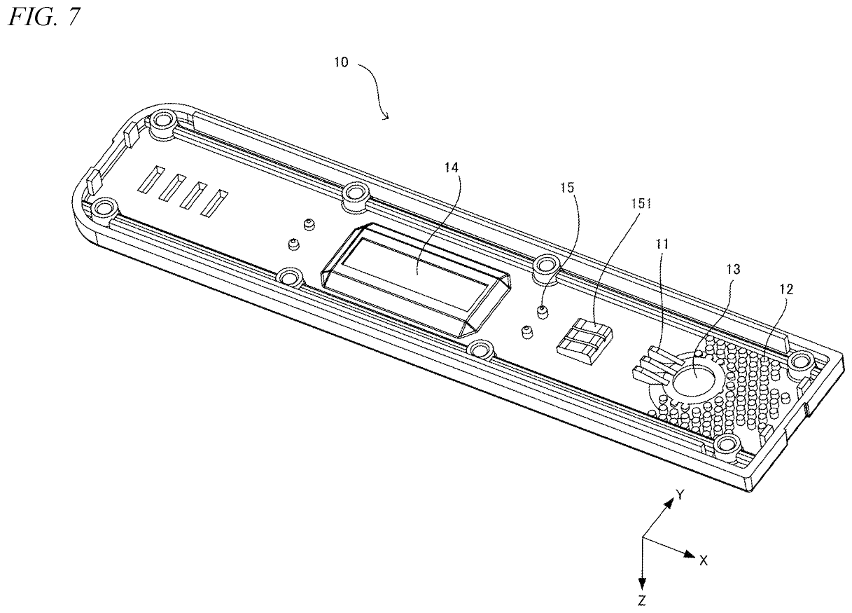

As to the pressing pins 15, two are disposed along the arranged direction X, and two are disposed along an orthogonal direction Y, which is orthogonal to the arranged direction X, i.e. four pressing pins 15 in total are disposed. The pressing pins 15 are members preventing the test strip 30 from floating upward. In FIG. 3, the pressing pins 15 are positioned at a predetermined distance from the test strip 30, but the pressing pins 15 may contact the test strip 30. However, the pressing pins 15 are not essential members, and the number, the positions, the shape, and the like of the pressing pins 15 are not particularly limited. Incidentally, in the present embodiment, the pressing pins 15 are provided on the upper case 10. However, the pressing pins 15 are not essential constituents but may be dispensed with. Moreover, in the present embodiment, each of the pressing pins 15 is provided in the shape of a pin but is not limited thereto. Pressing members 151 shown in FIG. 7 may be provided alternatively. The pressing members 151 may be configured to extend along the sectional shape of the test strip 30 to make overlapping among pads 22, 32 and 33 and a membrane 34 more stable.

The test strip 30 is a long member placed on the lower case 40. As shown in FIG. 3, the test strip 30 has a sample pad 31, the adjusting pad 32, the conjugate pad 33, the membrane 34, and the absorbent pad 22 that are provided sequentially along the arranged direction X. Incidentally, FIG. 3 schematically depicts the test strip 30 in which thicknesses of the adjusting pad 32, the conjugate pad 33, and the absorbent pad 22 are depicted to be not fixed. However, each of the sample pad 31, the adjusting pad 32, the conjugate pad 33, the membrane 34, and the absorbent pad 22 is actually formed into the shape of a sheet with a uniform thickness.

The sample pad 31 is exposed from the dropping hole 13. The sample pad 31 has a property of not only absorbing the liquid sample that has been added through the dropping hole 13 but also moving the liquid sample. In the present embodiment, the second protrusions 12 are provided adjacently to the dropping hole 13, and most of the liquid sample that has been added through the dropping hole 13 is pulled to the second protrusions 12 due to a capillary phenomenon, and then diffused into the sample pad 31. It is a matter of course that a portion of the liquid sample that has been added through the dropping hole 13 does not move toward the second protrusions 12 but moves toward the first protrusions 11 (details will be described later).

The adjusting pad 32 is disposed on the determination window 14 side of the sample pad 31 with a gap G provided therebetween. The adjusting pad 32 is a member disposed for the purpose of imparting a specific function to promote reaction of capturing an antigen contained in a liquid sample. An example of the specific function includes extraction etc. of the antigen contained in the liquid sample. Due to the gap G provided between the sample pad 31 and the adjusting pad 32, deterioration of a medicine infiltrated into the adjusting pad 32 can be suppressed. In a manner similar to or the same as the second protrusions 12, the first protrusions 11 are provided above this gap G adjacently to the dropping hole 13. Accordingly, the liquid sample can go along the first protrusions 11 from the sample pad 31 so as to move over the gap G toward the adjusting pad 32.

The conjugate pad 33 is disposed in continuity with the determination window 14 side of the adjusting pad 32. The conjugate pad 33 is partially laminated on the adjusting pad 32. The conjugate pad 33 carries a specific antibody (antibody labeled with a substance such as gold colloid or color latex; colored label), and the liquid sample that has reached the conjugate pad 33 infiltrates the membrane 34 while dissolving the specific antibody. On this occasion, the antigen contained in the liquid sample binds to the specific antibody.

The membrane 34 is disposed in continuity with the determination window 14 side of the conjugate pad 33 and can be visually recognized through the determination window 14 of the upper case 10 present on the upper side. To the membrane 34, a capture antibody is applied and immobilized in the shape of a line along the orthogonal direction Y. When an antigen (an object to be measured) that has been bound to the specific antibody is present in the liquid sample, the antigen and the capture antibody immobilized to the membrane 34 cause an antigen-antibody reaction so that the colored label appears as a signal on the membrane 34. Further, the membrane 34 is connected to the absorbent pad 22 placed on an end portion of a backing sheet 20 so that the liquid sample is finally absorbed by the absorbent pad 22.

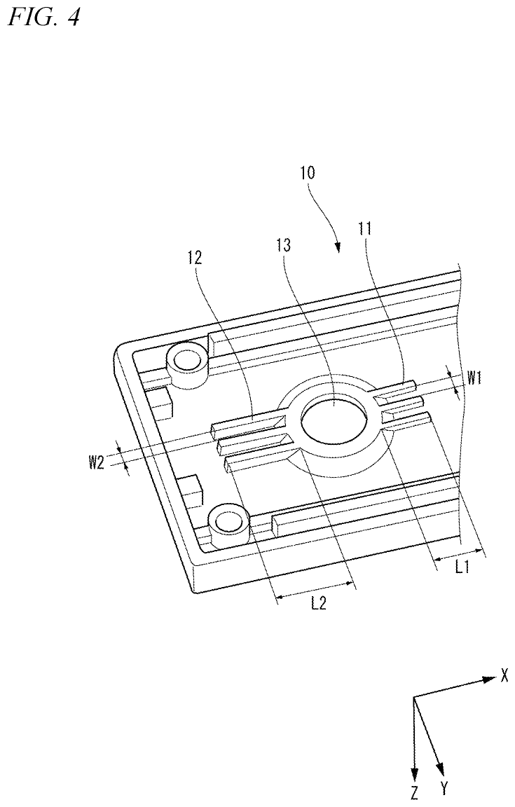

FIG. 4 shows an enlarged perspective view of a key part of the upper case 10 shown in FIG. 1. Each of the first protrusions 11 protrudes from the determination window 14 side of the dropping hole 13 toward the lower case 40, that is, protrudes along the thickness direction Z and is provided along the direction X in which the dropping hole 13 and the determination window 14 are arranged. In the present embodiment, the three first protrusions 11 each of which presents the shape of a strip extending like a straight line along the arranged direction X are disposed along the orthogonal direction Y.

On the other hand, each of the second protrusions 12 protrudes from the opposite side of the dropping hole 13 to the determination window 14 toward the lower case 40, that is, protrudes along the thickness direction Z and is provided along the arranged direction X. In the present embodiment, the three second protrusions 12 each of which presents the shape of a strip extending like a straight line along the arranged direction X are disposed along the orthogonal direction Y.

In the present embodiment, a length of each of the second protrusions 12 in the arranged direction X is longer than a length of each of the first protrusions 11 in the arranged direction X. That is, as shown in FIG. 4, the length L1 of the first protrusion 11 in the arranged direction X and the length L2 of the second protrusion 12 in the arranged direction X establishes a relation L1<L2.

According to such a configuration, capillary force generated between the second protrusions 12 and the liquid sample can be made larger than capillary force generated between the first protrusions 11 and the liquid sample. Thus, force with which the second protrusions 12 pull the liquid sample that has been dropped on the test strip 30 toward the opposite side to the determination window 14 can be made stronger than force with which the first protrusions 11 pull the liquid sample that has been dropped on the test strip 30 toward the determination window 14. That is, most of the liquid sample is spread to the side of the second protrusions 12 by the second protrusions 12, as shown by an arrow A in FIG. 3. The liquid sample that has spread to the side of the second protrusions 12 then moves toward the absorbent pad 22 by absorbing force of the absorbent pad 22. The liquid sample moving toward the absorbent pad 22 goes along the first protrusions 11 to climb over the gap G, and then passes through the conjugate pad 33 and the membrane 34 to be spread (infiltrated, made to flow) up to the absorbent pad 22. Incidentally, the shape, the structure, and the like of the test strip 30 are not particularly limited, but, for example, the adjusting pad 32 may be also dispensed with. Moreover, the adjusting pad 32 may be disposed between the conjugate pad 33 and the membrane 34.

Thus, the liquid sample testing device according to the present embodiment can control the flow of the liquid sample in the test strip 30 so that the liquid sample which has been dropped on the dropping hole 13 is once moved forward to the opposite side to the determination window 14 and then spread to the determination window 14 side. Thus, the liquid sample can be uniformly spread over the entire test strip 30. Moreover, due to the uniform spread of the liquid sample, a dry medicine that has been held by the test strip 30 can be sufficiently dissolved to improve efficiency of immune reaction and improve formation of a color indicating a test result.

Moreover, in the present embodiment, both the number of the first protrusions 11 and the number of the second protrusions 12 provided in the orthogonal direction Y orthogonal to the arranged direction X are plural (three in the present embodiment). An interval W2 between adjacent ones of the second protrusions 12 in the orthogonal direction Y is narrower than an interval W1 between adjacent ones of the first protrusions 11 in the orthogonal direction Y. That is, as shown in FIG. 4, the interval W1 between the adjacent first protrusions 11 in the orthogonal direction Y and the interval W2 between the adjacent second protrusions 12 in the orthogonal direction Y establish a relation W1>W2.

According to such a configuration, a capillary path formed between the adjacent second protrusions 12 is narrower than a capillary path formed between the adjacent first protrusions 11. Thus, the force with which the second protrusions 12 pull the liquid sample that has been dropped on the test strip 30 toward the opposite side to the determination window 14 can be made stronger than the force with which the first protrusions 11 pull the liquid sample that has been dropped on the test strip 30 toward the determination window 14. Therefore, the liquid sample can be spread uniformly in a manner similar to or the same as the aforementioned manner, and a pH adjuster, an aggregation inhibitor, salt, or the like (dry medicine) held by the test strip 30 can be sufficiently dissolved to improve the efficiency of the immune reaction and improve the formation of the color indicating the test result. Incidentally, the present embodiment has been described using the pH adjuster, the aggregation inhibitor, the salt, or the like, as an example of the dry medicine, but the dry medicine is not limited thereto. Any medicine can be used as the dry medicine as long as the medicine can be held by the test strip 30.

Since at least one of the aforementioned relations L1<L2 and W1>W2 is satisfied, the improvement in the formation of the color indicating the test result can be expected.

In addition, end portions 11b (see FIG. 3) of the first protrusions 11 on the dropping hole 13 side in the arranged direction X are located above the sample pad 31, and end portions 11a (see FIG. 3) of the first protrusions 11 on the determination window 14 side in the arranged direction X are located above the adjusting pad 32. According to such a configuration, the liquid sample that has been spread up to the end portions 11a on the determination window 14 side along the first protrusions 11 can be infiltrated into the adjusting pad 32. In addition, when the end portions 11a of the first protrusions 11 are extended to the conjugate pad 33, there is a fear that the liquid sample may go along a space between the first protrusions 11 and the test strip 30 to flow toward the membrane 34, thereby resulting in shallow infiltration into the adjusting pad 32. However, in the present embodiment, as described above, the end portions 11a of the first protrusions 11 are located above the adjusting pad 32. Accordingly, the liquid sample can be easily infiltrated into the adjusting pad 32.

In addition, the first protrusions 11 are provided to face the gap G but not inserted into the gap G. That is, the first protrusions 11 are in a state of covering the gap G. According to such a configuration, the liquid sample can flow along the first protrusions 11 to climb over the gap G so as to be spread up to the adjusting pad 32.

### Second Embodiment

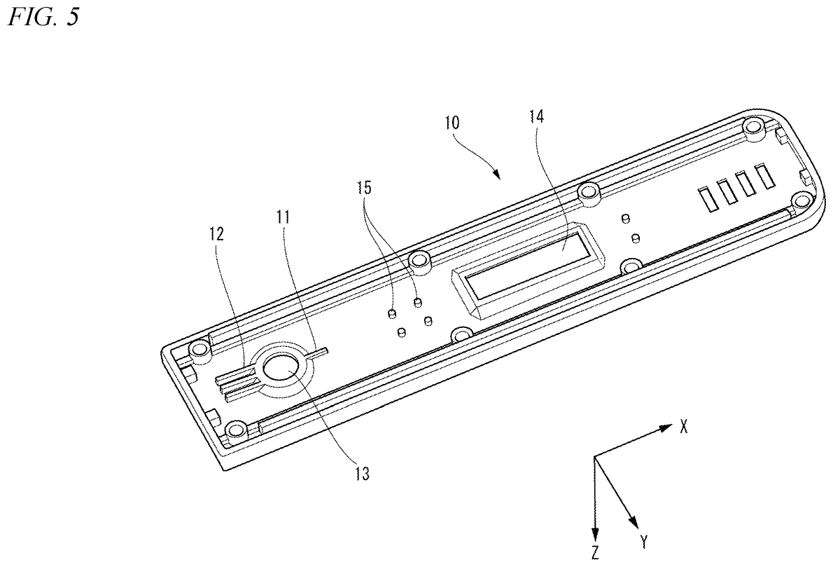

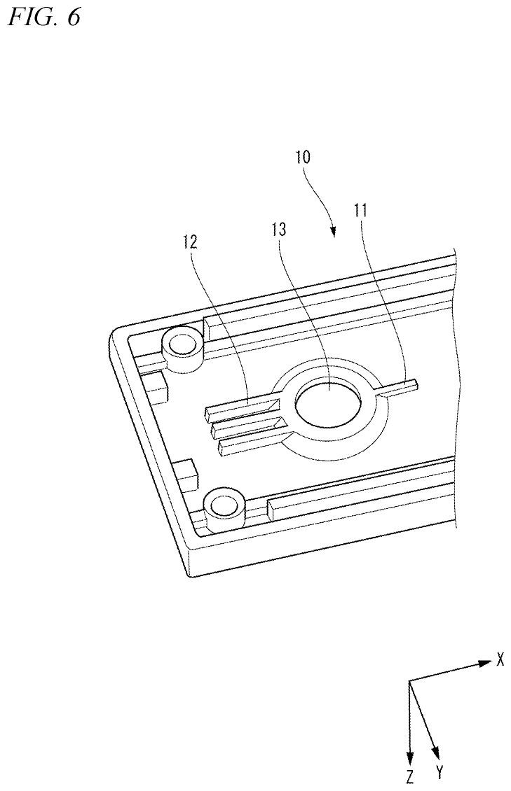

FIG. 5 shows a perspective view of an upper case 10 of a liquid sample testing device according to a second embodiment of the present invention. FIG. 6 shows an enlarged perspective view of a key part of the upper case 10 shown in FIG. 5. In the present embodiment, only one first protrusion 11 is provided, and three second protrusions 12 are provided in a manner similar to or the same as in the first embodiment. That is, the number of the second protrusions 12 is larger than the number of the first protrusions 11. The number of the first protrusions 11 and the number of the second protrusions 12 are not particularly limited. In the present embodiment, a relation (the number of the first protrusions 11<the number of the second protrusions 12) is established.

According to such a configuration, force with which the second protrusions 12 pull a liquid sample that has been dropped on a test strip 30 toward an opposite side to a determination window 14 can be made stronger than force with which the first protrusion 11 pulls the liquid sample that has been dropped on the test strip 30 toward the determination window 14. Therefore, the liquid sample can be spread uniformly in a manner similar to or the same as in the first embodiment, so that a dry medicine held by the test strip 30 can be dissolved sufficiently to improve efficiency of immune reaction and improve formation of a color indicating a test result.

### Third Embodiment

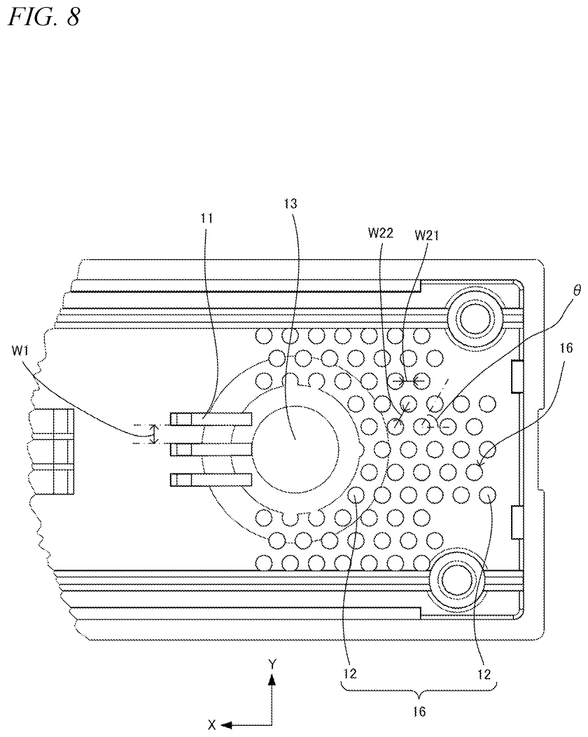

FIG. 7 shows a perspective view of an upper case of a liquid sample testing device according to a third embodiment of the present invention. FIG. 8 shows an enlarged top view of a key part of the upper case shown in FIG. 7. In the present embodiment, each of second protrusions 12 is provided in the shape of a cylinder (the pillar shape). The second protrusions 12 are provided to be spaced from one another along an arranged direction X from a peripheral portion of a dropping hole 13 toward an opposite side to a determination window 14. The second protrusions 12 aligned along the arranged direction X constitute second protrusion rows 16. The second protrusion rows 16 are provided to be arranged side by side in an orthogonal direction Y. In the present embodiment, the number of the second protrusion rows 16 provided to be arranged side by side is 11.

Incidentally, second protrusions 12 in one of the second protrusion rows 16 adjacent to each other and second protrusions 12 in the other second protrusion row 16 are not aligned in the orthogonal direction Y, but are aligned in a direction forming an angle θ of about 45° to 75° with respect to the arranged direction X. That is, each second protrusion 12 in one of the adjacent second protrusion rows 16 is located between two second protrusions 12 in the other second protrusion row 16. Thus, an interval W22 between the second protrusions 12 constituting the adjacent second protrusion rows 16 can be made narrower. An interval W21 between adjacent ones of the second protrusions 12 in the same second protrusion row 16 is either the same as or narrower than the interval W22. The intervals W21 and W22 of the second protrusions 12 are provided to be narrower than an interval W1 between adjacent ones of first protrusions 11.

In addition, in the present embodiment, second protrusion rows 16 each of which is constituted by six second protrusions 12 and second protrusion rows 12 each of which is constituted by five second protrusions 12 are aligned alternately in a total of five rows from the peripheral portion of the dropping hole 13 on the side far from the determination window 14. Further, three second protrusion rows 16 each of which is constituted by seven second protrusions 12 are arranged side by side from each of peripheral portions on opposite sides of the dropping hole 13 in the orthogonal direction Y. It is a matter of course that the positions or the numbers of the pillar-shaped second protrusions 12 disposed thus in the present invention are not limited to those in the example shown in FIGS. 7 and 8.

According to the aforementioned configuration, each of the intervals W21 and W22 between the adjacent second protrusions 12 is narrower than the interval W1 between the adjacent first protrusions 11 in the orthogonal direction Y in a manner similar to or the same as in the first embodiment. That is, a capillary path formed between the adjacent second protrusions 12 is narrower than a capillary path formed between the adjacent first protrusions 11. Thus, capillary force generated between the second protrusions 12 and the liquid sample can easily be made larger than capillary force generated between the first protrusions 11 and the liquid sample.

As described above, whether each of the second protrusions 12 has a shape extending in the arranged direction X as in the first embodiment or a pillar shape, the second protrusions 12 can generate stronger capillary force as the interval W2, W21, or W22 between adjacent ones of the second protrusions 12 is narrower. However, when the interval W2, W21, or W22 is made excessively narrow, there is a fear that a liquid amount for holding the liquid sample among the second protrusions 12 is insufficient. Therefore, the interval W2, W21 or W22 is preferably from 0.2 mm to 1.0 mm, more preferably from 0.3 mm to 0.7 mm.

Incidentally, the present invention is not limited to the aforementioned embodiments, but modification, improvement, and the like can be appropriately made thereon. Besides, the materials, the shapes, the dimensions, the numerical values, the forms, the numbers, the disposed places, and the like of the constituent elements in the aforementioned embodiments are not limited but may be determined desirably as long as they can achieve the present invention.

For example, each of the second protrusions 12 in the second embodiment is provided in the shape of the cylinder but is not limited thereto. The second protrusion 12 may be brush-shaped or porous.

In addition, in the second embodiment, the second protrusions 12 are also provided in the peripheral portions on the opposite sides of the dropping hole 13 in the orthogonal direction Y but are not limited thereto. The second protrusions 12 may be not provided in the peripheral portions on the opposite sides of the dropping hole 13 in the orthogonal direction Y.

Moreover, in order to more effectively adjust the capillary forces generated between the liquid sample and the first and second protrusions 11 and 12, respective surface conditions of the first protrusions 11 and the second protrusions 12 may be adjusted. According to specific methods, a microstructure of irregularities or the like is applied to the surfaces of the first protrusions 11 and the second protrusions 12 by formation of the first protrusions 11 and the second protrusions 12 or modification (such as coating) of their surfaces using a material having appropriate surface energy, or by blasting treatment, or the like. However, the adjustment of the surface conditions is not limited thereto as long as the capillary forces can be adjusted. The adjustment of the surface conditions does not have to be performed only on the surfaces of the first protrusions 11 and the second protrusions 12. The adjustment of the surface conditions may be performed on the other portion of the upper case 10 than the first protrusions 11 and the second protrusions 12 (e.g. the entire surface of the upper case 10 or the circumferences of the bases of the first protrusions 11 and the second protrusions 12 of the upper case 10).

The present application is based on a Japanese patent application (Patent Application No. 2019-135727) filed on Jul. 23, 2019 and a Japanese patent application (Patent Application No. 2020-113853) filed on Jul. 1, 2020, the contents of which are incorporated herein by reference.

### INDUSTRIAL APPLICABILITY

According to the liquid sample testing device in the present invention, the flow of the liquid sample in the test strip can be controlled so that the liquid sample can be uniformly spread over the entire test strip. In addition, due to the uniform spread of the liquid sample, the dry medicine held by the test strip can be sufficiently dissolved to improve the efficiency of the immune reaction and improve the formation of the color indicating the test result. The present invention obtaining this effect is useful for the liquid sample testing device.

### REFERENCE SIGNS LIST

10 upper case
11 first protrusion
12 second protrusion
13 dropping hole
14 determination window
15 pressing pin
20 backing sheet
22 absorbent pad
30 test strip
31 sample pad
32 adjusting pad
33 conjugate pad
34 membrane
40 lower case

1. A liquid sample testing device comprising:
an upper case having: a hole allowing a liquid sample to be dropped in the hole; and a window to determine a test result, the hole and the window being located beside each other;
a lower case assembled to the upper case; and
a test strip housed between the upper case and the lower case,
the upper case having: a first protrusion protruding toward the lower case from a peripheral portion of the hole, the first protrusion being located on a side near to the window of the hole; and a second protrusion protruding toward the lower case from the peripheral portion of the hole, the second protrusion being located on a side opposite to the window of the hole compared to the first protrusion,
the first protrusion and the second protrusion each having a predetermined shape to allow a capillary force generated between the second protrusion and the liquid sample to be larger than a capillary force generated between the first protrusion and the liquid sample.

2. The liquid sample testing device according to claim 1, wherein
the first protrusion extends from the peripheral portion of the hole toward the window along an arranged direction of the hole and the window,
the second protrusion extends from the peripheral portion of the hole to separate from the window along the arranged direction.

3. The liquid sample testing device according to claim 2, wherein
a length of the second protrusion in the arranged direction is longer than a length of the first protrusion in the arranged direction.

4. The liquid sample testing device according to claim 2, wherein
a plurality of the second protrusions are larger in number than a plurality of the first protrusions.

5. The liquid sample testing device claim 2, wherein
a plurality of the first protrusions are arranged side by side in a direction different from the arranged direction, and a plurality of the second protrusions are arranged side by side in a direction different from the arranged direction,
an interval between adjacent pair of the second protrusions is narrower than an interval between adjacent pair of the first protrusions.

6. The liquid sample testing device according to claim 1, wherein
the second protrusion has a pillar shape,
a plurality of the second protrusions are aligned at intervals from the peripheral portion of the hole to separate from the window along an arranged direction of the hole and the window, and the plurality of the second protrusions form a second protrusion row,
a plurality of the second protrusion rows are arranged side by side in a direction different from the arranged direction.

7. The liquid sample testing device according to claim 6, wherein
the first protrusion extends from the peripheral portion of the hole toward the window along the arranged direction,
a plurality of the first protrusions are arranged side by side in a direction different from the arranged direction,
an interval between adjacent pair of the second protrusions is narrower than an interval between adjacent pair of the first protrusions.

8. The liquid sample testing device according to claim 1,
the test strip has: a sample pad exposed in the hole; an adjusting pad located on a side near to the window of the sample pad with a gap between the sample pad and the adjusting pad; a conjugate pad located on a side near to the window of the adjusting pad, the conjugate pad touches to the adjusting pad; and a membrane located on a side near to the window of the conjugate pad, the membrane touches to the conjugate pad, the membrane is visually recognized through the window,
an end portion of the first protrusion on a side near to the hole in an arranged direction of the hole and the window is located above the sample pad, and an end portion of the first protrusion on a side near to the window in the arranged direction is located above the adjusting pad.

9. The liquid sample testing device according to claim 1,
the test strip has: a sample pad exposed in the hole; an adjusting pad located on a side near to the window of the sample pad with a gap between the sample pad and the adjusting pad; a conjugate pad located on a side near to the window of the adjusting pad, the conjugate pad touches to the adjusting pad; and a membrane located on a side near to the window of the conjugate pad, the membrane touches to the conjugate pad, the membrane is visually recognized through the window,
the first protrusion faces to the gap without being inserted into the gap.
